(19) United States
(12) Patent Application Publication
(10) Pub. No.: US 2003/0044754 A1
(43) Pub. Date: Mar. 6, 2003

(54) Direct bonding dental arch bar

(76) Inventors: Richard J. Deslauriers, Woodbury, CT (US); Robert T. Potash, South Windsor, CT (US); Stephanie Bonin, East Hartford, CT (US)

Correspondence Address: Richard J. Deslauriers, M.D., 50 Altair Avenue, Plymouth, CT, 06782, US

(21) Appl. No.: 09/947,001

(22) Filed: Sep. 5, 2001

(51) Int. Cl. A61C 5/00; A61F 5/00

(52) U.S. Cl. 433/215; 602/17

(57) ABSTRACT

A direct bonding dental arch bar comprised of a flexible base portion having a first side and a second side, which may be comprised of a woven medical grade nylon. This arch bar further comprises a dental positioning device, where said positioning device is an at least one guideline and locking lug, and may utilized to secure this arch bar to a patient's row of teeth prior to adhesion to the teeth is permitted. Furthermore, a light curable adhesive is used that enables said flexible base portion to be secured to at least one tooth when exposed to light, and in particular a blue light spectrum.

### BACKGROUND OF THE INVENTION

1. Field of the Invention

This invention relates to the field of maxillofacial fractures, and more particularly to direct bonding dental arch bars.

2. Description of the Related Art

The goals of maxillofacial fracture repair are to obtain good dental occlusion, achieve bony union, preserve mental and facial nerve function, prevent post-traumatic sequelae, and attain optimal cosmesis by retaining teeth and minimizing external scars. Additionally, the teeth should be brought into the best possible relationship so that adequate chewing surface and joint function occur with minimal or no orthodontic manipulation after bony reduction.

Certain devices that are used in the surgical reconstruction of maxillofacial fractures may be characterized as arch bars. These arch bars may be secured to a patient's maxilla and/or mandible, or the upper and/or lower teeth. Furthermore, these devices may utilize interdental wiring to secure the arch bar to one or more teeth of a patient's maxilla or mandible. The use of interdental wiring may provide for an increased risk in lacerating a patient's gums and mouth and, thereby causing bleeding in the patient's mouth. Additionally, the likelihood of puncturing the rubber gloves worn by the doctors in surgical procedures may be additionally increased, and which puncture may further cause the puncturing of the doctor's skin under his gloves. It is this double puncturing of both the patient and the doctor's protection that may increase the potential for the doctor to be infected with blood born pathogens in the patient's blood. Additionally, these devices that utilize interdental wiring may be difficult and time consuming to use as they may be difficult for the doctor to apply and secure the arch bar to the patient in a quick and efficient manner.

Other teachings provide for the application of direct bonding arch bars, and which arch bars may be secured to the patient's maxilla or mandible without circumdental wiring. However, these devices do not provide for a simplified and efficient manner in which the arch bar may be secured or stabilized to the teeth of the maxilla and/or mandible prior to the adhesive drying. This may prevent the device from staying stationary during the adhesive process and, as such, the movement of the device may cause increased time in application and may further decrease the bonding strength of the arch bar to the maxilla or the mandible. Additionally, many of these devices are manufactured using a metal mesh, in which the application of the metal mesh proves to be difficult to achieve high bonding strength to the patient's teeth.

### BRIEF SUMMARY OF THE INVENTION

Therefore a need has arisen for a maxillofacial dental arch bar that overcomes these and other shortcomings of the related art. A technical advantage of the present invention is to provide a direct bonding arch bar that does not require the use of circumdental wiring to affix the device to the teeth adjacent to either the maxilla or the mandible, wherefrom the danger of wires puncturing gloves and gums may be eliminated. A further technical advantage is that this device may provide for a means of securing placement or stabilizing a dental arch bar prior to adhesion. Another technical advantage of the present invention is that the device may prevent any movement of the fragmented bone around the fracture sight, wherein bone healing may be maximized and infection may be minimized. Another technical advantage of the present invention is that operating time and postoperative patient discomfort may be minimized. Yet another technical advantage is that the device may allow for quick and painless application and removal, further reducing the traumatic effects on the patient's maxilla or mandible, and may increase patient comfort.

In an embodiment of the present invention, a direct bonding dental arch bar is described. The arch bar may comprises a flexible base portion having a first side and a second side, wherein said base portion may comprise a woven medical grade nylon, a dental positioning device, wherein said positioning device may be an at least one guideline and locking lug, and an adhesive that enables said flexible base portion to be secured to at least one tooth. In an exemplary embodiment, dental arch bar may comprise a flexible base portion manufactured from medical grade nylon.

In still yet another embodiment the dental arch bar may further be comprised of a dental positioning device, which positioning device may be comprised of a guideline used in connection with a locking lug. Furthermore, the flexible base portion may utilize interwoven fixation clips extending through the second side of said flexible base portion, and wherein said fixation clips may be utilized for vertical oriented guide wires.

Other objects, features, and advantages will be apparent to persons of ordinary skill in the art in view of the following detailed description of the invention and the accompanying drawings.

### BRIEF DESCRIPTION OF THE SEVERAL VIEWS

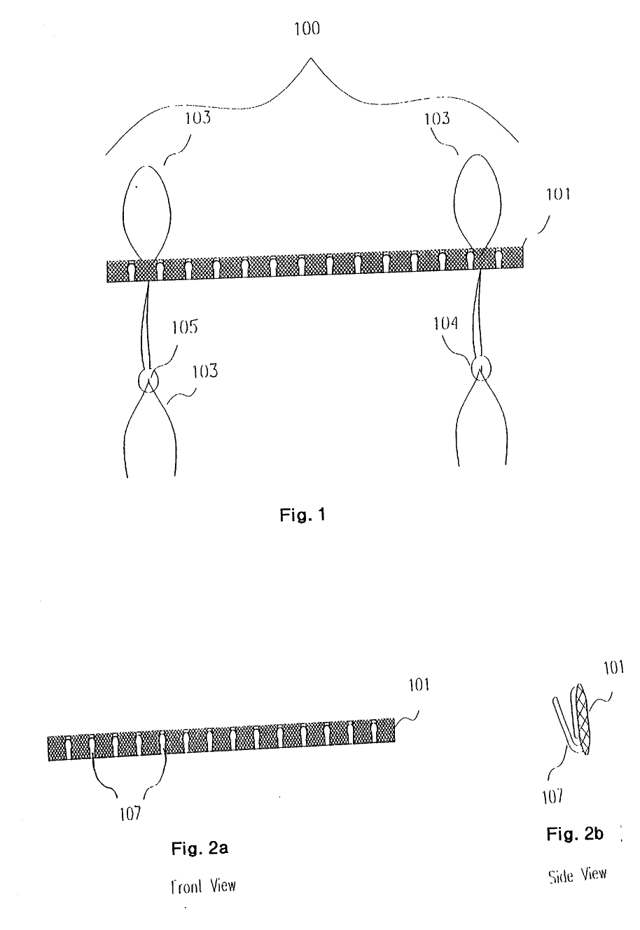

FIG. 1 shows a front view of the direct bonding dental arch bar according to an embodiment of the present invention.

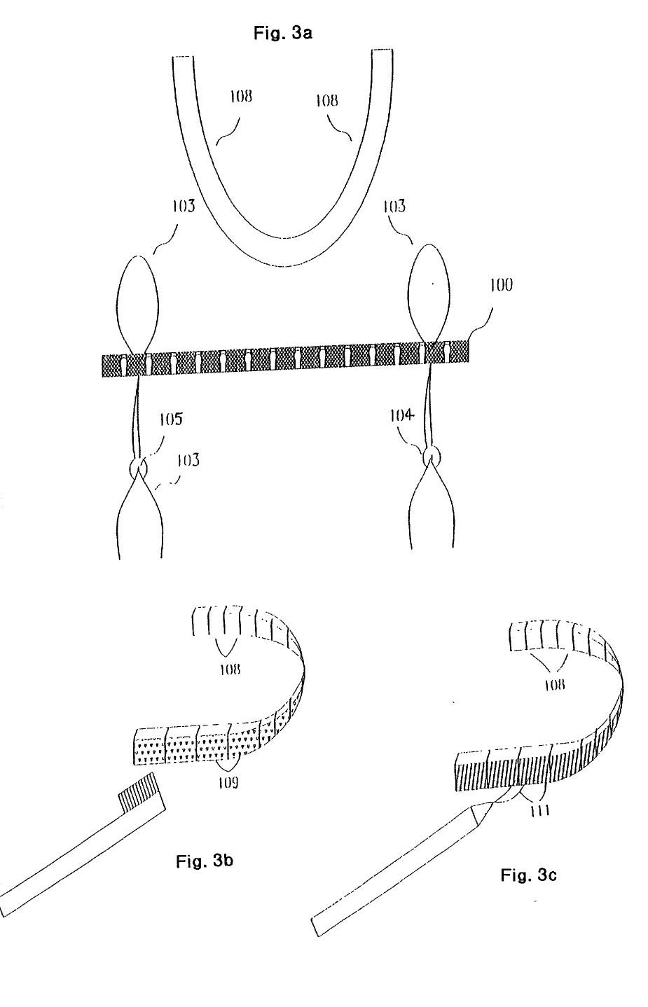

FIG. 2a shows a front view of the flexible base portion according to an embodiment of the present invention.

FIG. 2b shows a side view of the flexible base portion according to an embodiment of the present invention.

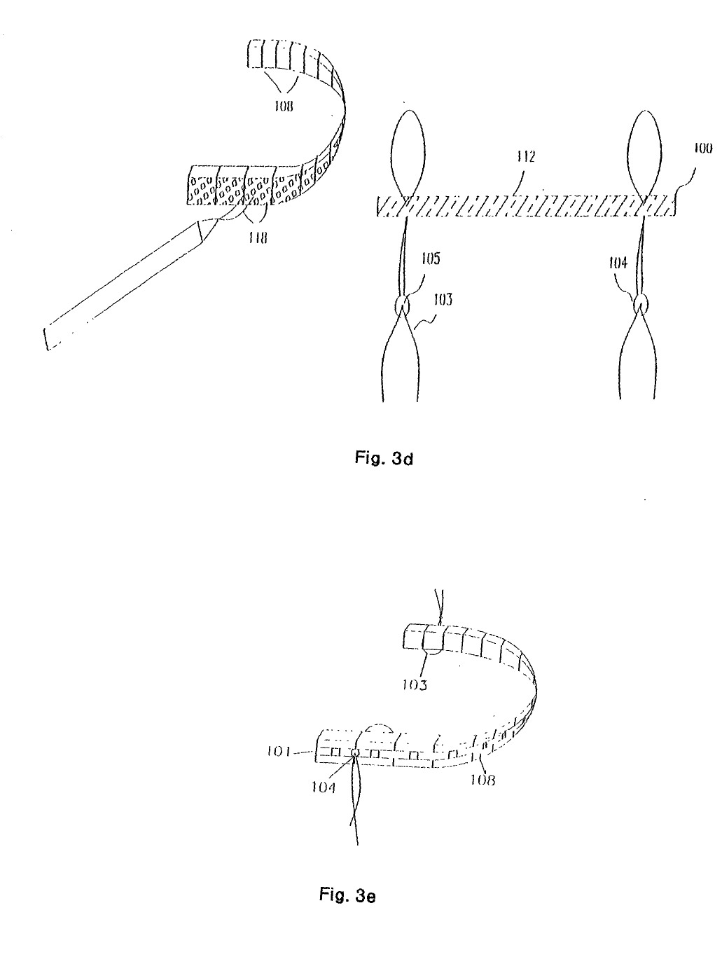

FIG. 3a depicts the method of use according to the preferred embodiment of the present invention.

FIG. 3b depicts the method of use according to the preferred embodiment of the present invention.

FIG. 3c depicts the method of use according to the preferred embodiment of the present invention.

FIG. 3d depicts the method of use according to the preferred embodiment of the present invention.

FIG. 3e depicts the method of use according to the preferred embodiment of the present invention.

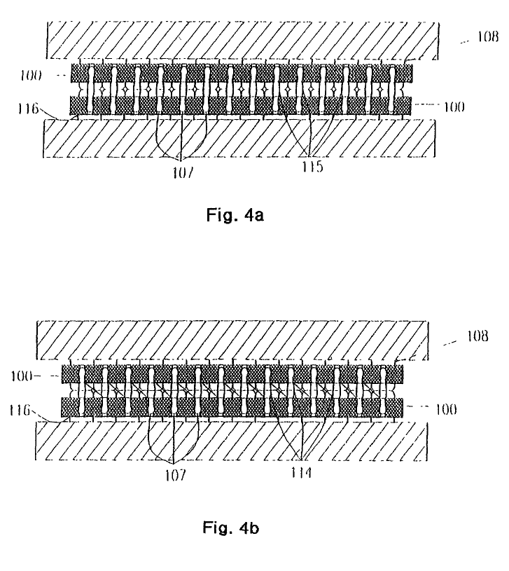

FIG. 4a depicts the method of use according to another embodiment of the present invention.

FIG. 4b depicts the method of use according to another embodiment of the present invention.

### DETAILED DESCRIPTION OF THE INVENTION

Preferred embodiments of the present invention and their advantages may be understood by referring to FIGS. 1-4b, like numerals being used for like corresponding parts in the various drawings.

Referring to FIG. 1, a direct bonding dental arch bar 100 according to a preferred embodiment of the present invention is described. Arch bar 100 according to an embodiment of the present invention may serve as a means of securing the teeth of the mandible or maxilla of a patient in a fixed position such that fragmented bone of either the maxilla or mandible may be maintained in a fixed position. Arch bar 100 may be comprised of a flexible base 101, dental positioning device, and an adhesive. Base portion 101 may be manufactured from material from a group of materials consisting of medical grade nylon, Kevlar®, woven metal mesh, or the like. In an embodiment, flexible base 101 may be of a width of about 4.6 mm. In another embodiment, the width of flexible base 101 may be no greater than about 8.5 mm. Further according to an embodiment of the present invention, flexible base 101 may be manufactured so as to be impregnated or coated with a general adhesive, whereas the general adhesive may provide for a preliminary method of securing arch bar 100 to the teeth of a patient, and wherein said adhesive may further be light curable.

In an embodiment of this invention positioning device may be comprised of guideline 103, which guideline 103 may be of a medical grade nylon, such as dental floss, and whereas dental positioning device may further be comprised of locking lug 104. Guideline 103 and locking lug 104 may interact together, such that guideline 103 may looped and inserted into slide catch 105, whereas slide catch 105 may provide a means for securing guideline 103 at a designated position within locking lug 104, and wherefrom the securing of guideline 103 may provide for a loop that upon being placed in slide catch 105 may provide for a method of retaining the position of guideline 103.

Referring to FIGS. 2a and 2b, another embodiment of the present invention is shown depicting fixation clips 107. Fixation clips 107 may be comprised of a medical grade stainless steel, titanium, plastic, or the like, and whereas fixation clips 107 may be manufactured in a design so as to provide for multiple support elements that may extend through the second side of said flexible base portion 101. According to this embodiment of the present invention, fixation clips 107 may be utilized for securing vertical oriented guide wires to promote stabilization of the mandible relative to the maxilla.

In another embodiment (not shown), dental positioning device may comprise an elongated handle, wherein said handle may be attached to said flexible base portion 101, and wherein said handle may be used to stabilize and retain said base portion 101 in a desired location.

FIGS. 3a- 3e show the preferred method of securing arch bar 100 to at least one row of teeth 108 according to this invention. A method of securing an at least one direct bonding dental arch bar 100 to an least one row of teeth 108 may be comprised of first removing excess plaque 109 from the exterior tooth surface of a first row of teeth 108, wherein plaque 109 removal may be facilitated through the utilization of prophylaxis paste applied to teeth 108 with a standard type toothbrush 117. After plaque 109 has been removed from teeth 108, then guideline 103 may be placed circumdentally around a tooth on said first row of teeth 108.

Subsequent to attaching guidelines 103, an acid etching gel 111 may be used on the outer surface of teeth 108, which may then provide for micro-mechanical retention, wherein acid etching gel 111 may be of a material such as phosphoric acid etchant. In another embodiment, teeth 108 may be cleansed further in another step, wherein chlorohexadine gluconate may be used on teeth 108, thereby further removing any remaining substances on the surface of teeth 108.

Following the application of etching gel 111 and after teeth 108 have been substantially dried, guidelines 103 may be used to tie down the posterior ends of said arch bar 100, wherein said guidelines may be secured around the secondary premolars of said patient. In an embodiment, teeth 108 may be dried, wherein compressed nitrogen may be applied to the exterior surface to facilitate the complete drying of teeth 108.

An adhesive primer 118 may be applied to the outer surface of dry teeth 108 along with the application of curable adhesive 112 to the first side of arch bar 100. Curable adhesive 112 may be an adhesive that is light curable, and more specifically, said curable adhesive 112 might be particularly responsive from the application of blue light. In another embodiment, arch bar 100 may be pre-impregnated with adhesive 112.

After teeth 108 have been properly primed with primer 118 and curable adhesive 112 applied to arch bar 100, then arch bar 100 may be placed across the patient's teeth, such that said flexible base 101 extends from the first molar on one side of patient to the first molar on a second side of the patient. Additionally, arch bar 100 may retain its initial desired position before being permanently cured, since primer 118 and adhesive 112 may begin to cure immediately upon being exposed to any light.

According to this method of application, after guideline 103 has mechanically secured arch bar 100 to teeth 108, then blue light 113 may be used to cure and harden said adhesive 112, whereby blue light 113 may be applied uniformly over the entire length of flexible base 101 and facilitating rapid and secure bonding.

Referring to FIGS. 4a-4b, another embodiment of the present method is described wherein a second arch bar 100 may be secured to a second row of teeth 116 in accordance with the method described herein and depicted in FIGS. 3a-3e.

Referring to FIGS. 4a, another embodiment of the present invention, a method comprising securing a first arch bar 100 vertically to second arch bar 100 is described, wherein said method may vertically and horizontally stabilize a mandible and maxilla. First arch bar 100 and second arch bar 100 may be vertically attached through the use of integrated fixation clips 107, whereby a flexible wire 114 may be used to secure the fixation clips 107 of the first arch bar 100 to the second set of fixation clips 107 of the second arch bar 100.

Alternatively, as shown in FIG. 4b, the first set of fixation clips 107 may be vertically secured to the second set if fixation clips 107 through the use of heavy elastic bands 115. The vertical attachment of the first and second fixation clips may therefore provide for a method of establishing immobility of the first set of teeth 108 relative to the second set of teeth 116.

While the invention has been described in connection with preferred embodiments, it will be understood by those of ordinary skill in the art that other variations and modifications of the preferred embodiments described above may be made without departing from the scope of the invention. Other embodiments will be apparent to those of ordinary skill in art from a consideration of the specification or practice of the invention disclosed herein.

What is claimed is:

1. A dental arch bar comprising:
a flexible base portion;
dental positioning device; and
an adhesive,
wherein said flexible base comprises a first side and a second side, wherein said flexible base portion is stabilized to at least one tooth through said dental positioning system, and wherein said first side is secured to and across at least one tooth through said adhesive, and whereas said arch bar is used to stabilize structures in and around the mandible and maxilla.

2. The dental arch bar of claim 1, whereas said flexible base portion is comprised of a material selected from medical grade nylon.

3. The dental arch bar of claim 1, whereas said flexible base portion is comprised of a material selected from Kevlar®.

4. The dental arch bar of claim 1, whereas said flexible base portion is comprised of a material selected from fine metal mesh.

5. The dental arch bar of claim 1, whereas said flexible base portion is comprised of a combination of material selected from nylon, fine metal mesh, and Kevlar®.

6. The dental arch bar of claim 1, whereas said flexible base portion is of a width of about 4.6 mm.

7. The dental arch bar of claim 1, whereas said flexible base portion is of a width being about no greater than 8.5 mm.

8. The dental arch bar of claim 1, whereas said flexible base portion is pre-impregnated with said adhesive.

9. The dental arch bar of claim 8, whereas said adhesive is selected from a group of materials comprising a light curable adhesive, or a thixotropic medical device adhesive.

10. The dental arch bar of claim 1, whereas said flexible base portion incorporates an at least one fixation clip, and whereas said fixation clip secure vertically oriented guides, whereas said guides act for vertical stabilization of said arch bar to a second arch bar.

11. The dental arch bar of claim 10, whereas said fixation clips extend from said second side of said flexible base portion.

12. The dental arch bar of claim 10, whereas said fixation clips are manufactured from a group comprising plastic, titanium, or medical grade stainless steel.

13. The dental arch bar of claim 1, whereas said dental positioning device is comprised of a guideline used in connection with a locking lug.

14. The dental arch bar of claim 13, whereas said guideline is selected from a medical grade nylon thread.

15. The dental arch bar of claim 13, whereas said locking lug utilizes a slide catch to retain said guideline in a fixed position.

16. The dental arch bar of claim 1, whereas said dental positioning device is comprised of a guideline used in connection with a locking clamp.

17. The dental arch bar of claim 1, whereas said adhesive comprises a light curable adhesive, or a thixotropic medical device adhesive.

18. The dental arch bar of claim 1, whereas said light curable adhesive is comprised of an acrylated urethane.

19. An dental arch bar comprised of:
a flexible base portion having a first side and a second side, which is comprised of a woven medical grade nylon;
a dental positioning device, where said positioning device is an at least one guideline and locking lug; and
an adhesive that enables said flexible base portion to be secured to at least one tooth, and whereas said adhesive is acrylated urethane.

20. The dental arch bar of claim 19, whereas said flexible base portion utilizes interwoven fixation clips for vertical oriented guide wires.

21. The dental arch bar of claim 19, whereas said flexible base portion is of a width of about 4.6 mm.

22. The dental arch bar of claim 19, whereas said flexible base portion is of width being no greater than 8.5 mm.

23. A method of securing an at least one direct bonding dental arch bar, of claim 19, to an least one row of teeth of a patient comprised of:
Using an acid etching to produce an etching pattern in said outer teeth's surface for micro-mechanical retention;
Drying the external surface of said patient's teeth;
Utilizing a dental positioning device to stabilize said arch bar across said teeth;
Applying adhesive to the first side of said dental arch bar;
Placing said dental arch bar across the patient's teeth, such that said brace extends from a first tooth on one side of patient's row of teeth to a second tooth of the patient's row of teeth; and
Curing said adhesive.

24. The method of claim 23, wherein dental positioning system comprises a guideline and a locking lug, wherein the posterior ends of said dental arch bar are mechanically secured to a first premolar and a second premolar prior to the application of said primer through the tightening of said guideline and secured with said locking lug.

25. The method of claim 23, whereas acid etching is accomplished through the application of a phosphoric acid etchant.

26. The method of claim 23, wherein said drying step comprises the application of compressed nitrogen to the exterior surface of said row of teeth.

27. The method of claim 23, wherein, prior to the connection of said guidelines to said first premolar and to said second premolar, said teeth are further stripped of external substances with a cleansing rinse.

28. The method of claim 27, wherein said cleansing rinse is comprised of Chlorohexadine Gluconate.

29. The method of claim 23, where said excess plaque is removed from the exterior surface of said teeth prior to the application of said acid etching, whereas adhesion of said arch bar to teeth is enhanced.

30. The method of Clam 23, wherein a primer to the outer tooth surface may be applied to further enhance the adhesive strength of the bond between said arch bar and said teeth.

31. The method of claim 23, whereas said method is utilized to secure a second direct bonding dental arch bar to a second row of teeth, wherein said first dental arch bar comprises a first set of fixation clips, wherein said second arch bar comprises a second set of fixation clips, and wherein said first set of fixation clips are vertically secured to said second set of fixation clips.

32. The method of claim 32, wherein said first arch bar is secured to said second arch bar with a material comprising a flexible wire, heavy elastic bands, or thread.
